(19) United States
(12) Patent Application Publication
(10) Pub. No.: US 2024/0017258 A1
(43) Pub. Date: Jan. 18, 2024

(54) HYDRODYNAMICALLY-INDUCED DROPLET MICROVORTICES FOR MODULATING CELL DYNAMICS

(71) Applicant: THE REGENTS OF THE UNIVERSITY OF CALIFORNIA, Oakland, CA (US)

(72) Inventors: Abraham P. Lee, Irvine, CA (US); Xuhao Luo, Irvine, CA (US); Braulio Cárdenas Benítez, Irvine, CA (US)

(21) Appl. No.: 18/353,005

(22) Filed: Jul. 14, 2023

Related U.S. Application Data
(60) Provisional application No. 63/389,676, filed on Jul. 15, 2022.

(51) Int. Cl. B01L 3/00

(52) U.S. Cl. CPC B01L 3/502784; B01L 2300/06

(57) ABSTRACT

A microfluidic platform comprising an inlet, a fluidic chamber, one or more trapping arrays, each trapping array comprising pillars separated by gaps, an outlet, and a droplet generator fluidly coupled to the inlet. The droplet generator may accept one or more particles and output particle-laden droplets comprising a particle surrounded by an aqueous solution surrounded by a carrier oil. The particle-laden droplets directed from the droplet generator, through the inlet, to the fluidic chamber, may be immobilized by the one or more trapping arrays. The droplet generator may generate a continuous flow of carrier oil through the inlet and through the fluidic chamber. The continuous flow of carrier oil may induce one or more microvortices at the one or more trapping arrays.

### CROSS-REFERENCES TO RELATED APPLICATIONS

This application is a non-provisional and claims benefit of U.S. Provisional Application No. 63/389,676 filed Jul. 15, 2022, the specification of which is incorporated herein in their entirety by reference.

### STATEMENT REGARDING FEDERALLY SPONSORED RESEARCH OR DEVELOPMENT

This invention was made with government support under Grant No. IIP-1841509 awarded by the National Science Foundation. The government has certain rights in the invention.

### FIELD OF THE INVENTION

The present invention is directed to a microfluidic device for generating well-defined and configurable microvortices for the analysis of cells and cell-cell pair relationships.

### BACKGROUND OF THE INVENTION

Cell-cell interaction studies are important components in many biological processes, including genetics, oncology, and immunology. The hallmark of a successful immune system is orchestrated by a complex network of different types of cells that work in synergy to protect against foreign invaders. The study of specific interactions in single-cell biology requires high throughput and a microenvironment without interference from the background and neighboring cell pairs. While conventional co-culturing enables large quantities of cell-cell interactions, the lack of ability to control a number of interacting cells, interacting duration, and strength in a shared environment compromises the sensitivity and complexity of cell-cell interaction studies.

Droplet compartmentalization of cells has led to the development of single-cell analysis technologies, spanning from sequencing methods to cell-pairing microfluidic technologies, which have impacted the design and study of checkpoint blockade and adoptive T-cell transfer therapies. Preliminary results indicate that co-encapsulation of effector immune cells with their target enables real-time immunometabolic function assessment through glycolytic state determination via NADH autofluorescence. Notable microfluidic devices to study immune cell interactions include hydrodynamic traps that bring cells into close contact, and devices that actively control cell position through actuation (e.g. with microelectrodes). While droplet-based assays comparatively possess a throughput advantage because they are not restricted to a set number of traps per device, they cannot directly control cell pairing position and interaction frequency.

Microfluidic technology has been a powerful platform for analyzing cell-cell interactions at single-cell resolution. Notable microfluidic devices to study immune cell interactions include hydrodynamic traps that bring cells into close contact, and devices that actively control cell position through actuation (e.g. with microelectrodes). These techniques rely on the physical properties of cells or require complex fabrications that limit the amount of cell-cell interaction studies per device. Furthermore, high-density trap arrays have been demonstrated for high-throughput screening, and devices have been built where droplets can be individually addressed and retrieved for downstream analysis. However, when trapped droplets are considered for their use as controllable microreactors, the extent to which inner flow can be actuated through externally applied conditions remains largely unexplored. Droplet-based microfluidics comparatively possess a throughput advantage because they are not restricted to a set number of traps per device, they cannot directly control cell pairing position and interaction frequency. Although new technologies have emerged, the ability to modulate cell-cell interactions at scale for isolating specific interactions remains challenging. Thus, there is a need for the development of a versatile compartmentalization platform that maximizes throughput while augmenting the ability to modulate cell-cell interactions.

### BRIEF SUMMARY OF THE INVENTION

It is an objective of the present invention to provide systems and methods that allow for generating well-defined and configurable microvortices for the analysis of cells and cell-cell pair relationships, as specified in the independent claims. Embodiments of the invention are given in the dependent claims. Embodiments of the present invention can be freely combined with each other if they are not mutually exclusive.

The present invention features a microfluidic device that is capable of trapping cell-laden droplets and modulating the internal viscous stress to create microvortices of well-defined shape and periodicity, which enables the control of cell-cell distances of encapsulated cell pairs, as well as the high-throughput control of cell dynamics at the single level within droplets in two types of motion: orbiting and self-rotation (spinning). As an example, the present invention could be used to study juxtacrine and paracrine signaling by leveraging the microvortices in droplets to control contact frequency and distance between cells. With the dynamic controlling of the self-rotation of a cell in the droplet, this device would allow for examining cell morphology and surface markers without any active elements. This would be useful for applications where one wishes to study specific real-time cell-cell interactions and biomechanics within an isolated compartment at scale and prevent cross-interference.

The present invention features a microfluidic system comprising a microfluidic platform. In some embodiments, the microfluidic platform may comprise an inlet, a fluidic chamber fluidly coupled to the inlet, comprising one or more trapping arrays, each trapping array comprising a plurality of pillars separated by gaps, and an outlet fluidly coupled to the fluidic chamber. The microfluidic system may further comprise a droplet generator fluidly coupled to the inlet. The droplet generator may accept one or more cells and output one or more cell-laden droplets, each cell-laden droplet comprising a cell of the one or more cells surrounded by an aqueous solution surrounded by a carrier oil. The one or more cell-laden droplets directed from the droplet generator, through the inlet, to the fluidic chamber may be immobilized by the one or more trapping arrays. The droplet generator may generate a continuous flow of carrier oil through the inlet and through the fluidic chamber. The continuous flow of carrier oil may induce one or more microvortices at the one or more trapping arrays.

The present invention features a method providing a microfluidic system comprising a droplet generator and a microfluidic platform comprising an inlet, a fluidic chamber fluidly coupled to the inlet, and an outlet fluidly coupled to the fluidic chamber. The fluidic chamber may comprise one or more trapping arrays, each trapping array comprising a plurality of pillars separated by gaps. The method may further comprise accepting, by a droplet generator, one or more cells, generating, by the droplet generator, one or more cell-laden droplets, each cell-laden droplet comprising a cell of the one or more cells surrounded by an aqueous solution, surrounded by a carrier oil, and directing the one or more cell-laden droplets through the inlet of the microfluidic device to the fluidic chamber. The method may further comprise immobilizing, by the one or more trapping arrays, the one or more cell-laden droplets, and directing, by the droplet generator, a continuous flow of carrier oil through the inlet and through the fluidic chamber. The continuous flow of carrier oil may induce one or more microvortices at the one or more trapping arrays.

Two types of prior microfluidic devices have led to microvortices inside droplets in the literature: i) passive microfluidic devices, in which recirculation flows inside droplets arise due to droplets moving relative to their surrounding medium (i.e. slit channels, winding channels, and serpentine channels). The majority of these devices have been used to increase the mixing efficiency of reagents inside encapsulated droplets, but not for controlling cell movement inside them for pairing applications; ii) Microfluidic trap arrays, which include static hydrodynamic traps, pressure-actuated traps, microcage arrays, two-layer vertical hydrodynamic traps, and Hele-Shaw microfluidic vertical traps. In trap arrays, the focus has been mostly to observe trapped droplets and their contents (cells, reagents, etc.). However, none of the aforementioned applications have the hydrodynamic trap to allow lateral flow while holding droplets in place as in the present invention. Thus, the device allows for control of: 1) the microvortices created inside droplets; 2) the shape of the microvortices, which impacts how objects such as particles or cells, travel within them; 3) the recirculation time of encapsulated particles (i.e. the time it takes for an object to complete one loop inside a vortex).

One of the unique and inventive technical features of the present invention is the combination of specialized trapping arrays comprising pillars separated by gaps and specialized droplets comprising cells surrounded by an aqueous solution surrounded by a carrier oil. Without wishing to limit the invention to any theory or mechanism, it is believed that the technical feature of the present invention advantageously provides for leveraging the shear stress at the liquid-liquid interface to induce microvortices of well-defined shape and periodicity for cell pairing applications. None of the presently known prior references or work has the unique inventive technical feature of the present invention.

Furthermore, the inventive technical feature of the present invention contributed to a surprising result. For example, one skilled in the art would expect that external materials or components would be required to generate a 3D rotation pattern of cells in a microvortex generation device. Surprisingly, the present invention implements a specific trap shape, as well as the relative size of the encapsulated particle/cell to droplet size, to affect the observed particle movement within them and to control between particle 3D self-rotation (spinning), and orbiting (i.e. particles traveling in loops within the droplets). No other device in the literature has reported this type of behavior in a hydrodynamic trap.

In a non-limiting embodiment, the specialized trap for cell-laden droplets may comprise pillars separated by one central gap and two lateral gaps such that the cell-laden droplet blocks the central gap and carrier oil runs through the two lateral gaps to the side of the droplet. Without wishing to limit the invention to any theory or mechanism, it is believed that the technical feature of the present invention advantageously provides for oil to continue to be perfused into the chip, contacting the sides of the droplet as it flows past it and transferring viscous shear. The shear at the surface of the droplet creates recirculation inside it, thus forming the microvortices. This trap design enables the preservation of liquid-liquid interface between the aqueous solution and the carrier oil, such that the external oil flow can exert shear induced microvortices inside a droplet. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a schematic of the hydrodynamic droplet cell-cell pairing device of the present invention. FIG. 1B shows a flow chart of the method for immobilizing cell-laden droplets and inducing microvortices of well-defined shape and periodicity.

FIG. 2A shows a bright-field image of a trapping array with droplets encapsulating K562 cell pairs. FIG. 2B shows a schematic of dispersed and continuous phase flow streamlines of a trapped droplet. FIG. 2C shows a velocity flow field of continuous phase around a trapped droplet.

FIG. 3A shows streamlines inside and outside of droplets. FIG. 3B shows a K562 cell circulating in a microvortex. FIG. 3C shows the particle tracking velocimetry (PTV) of a K562 cell circulating in a microvortex. Overlayed in this image, is a graph of the time-dependent path taken by the circulating cell, r(t). FIG. 3D shows the Fourier transform of r(t). FIG. 3E shows time-course plots of the cell polar coordinates r(t), θ(t). FIG. 3F shows the measured recirculation time period T vs. external phase flow rate for the cell of FIG. 3B.

FIG. 4A shows two encapsulated K562 cells circulating (orbiting) in microvortices. FIG. 4B shows the time-courses of the magnitude and phase of the compound position vector. FIG. 4C shows a Fourier transform of the time-course of the compound position vector of FIG. 4B.

FIG. 5A shows two encapsulated K562 cells self-rotating in the center of microvortices. FIG. 5B shows the measured rotation time T vs. external phase flow rate of the cells depicted in FIG. 5A.

FIGS. 6A-6C show a schematic of the complete 3D volumetric single-cell reconstruction process.

FIG. 7 shows a close-up view of trapped droplets with rotating K562 cells.

FIG. 8 shows a graph showing linear dependence of the normalized cell angular velocity vs. the applied flow rate (tested at 1, 2, 5 and 10 μLmin−1, N=3).

FIG. 9 shows a comparison of 3D volumetric reconstructions of the surface of a K562 cell between the present invention and Structured Illumination Microscopy using z-axis optical sectioning. Left: The top row shows the maximum projection intensity of immune cell stained for CD45 surface marker and a 3D surface model for the image on the left (isosurface). The bottom row shows a raw image of a rotating cell, highlighting the nucleus, and a reconstruction 3D surface model of the nucleus from the image on the left. Right: 3D volumetric reconstructions of the surface of a K562 cell through the use of Structured Illumination Microscopy using z-axis optical sectioning, showing significant noise and artifacting compared to the present invention.

FIG. 10 shows a graph of the cell-size vs. droplet-size ratio effects on spinning and orbiting mechanisms.

### DETAILED DESCRIPTION OF THE INVENTION

Following is a list of elements corresponding to a particular element referred to herein:
100 microfluidic system
110 microfluidic platform
112 inlet
114 fluidic chamber
116 outlet
118 trapping array
120 droplet generator

The term “epifluorescence microscopy” is defined herein as a form of imaging, wherein the illumination beam penetrates the full depth of the sample, allowing easy imaging of intense signals and co-localization studies with multi-colored labeling on the same sample.

The term “optical projection tomography” is defined herein as a method of tomography, wherein a series of optics focuses illumination onto the sample and then the fluorescent light is directed to a camera. In some embodiments, the camera may the continuous flow of carrier oil has a flow rate such that the one or more microvortices induce, for each particle-laden droplet of the one or more particle-laden droplets, spinning of the particle within the particle-laden droplet or orbiting of the particle around a point within the particle-laden droplet.

comprise a charged-coupled device (CCD) camera, a complementary metal oxide semiconductor (CMOS) camera, or a scientific CMOS camera. An image is acquired at a series of angles and tomographic reconstruction is performed using a back-projection algorithm, and this yields a 3D volumetric representation of the specimen.

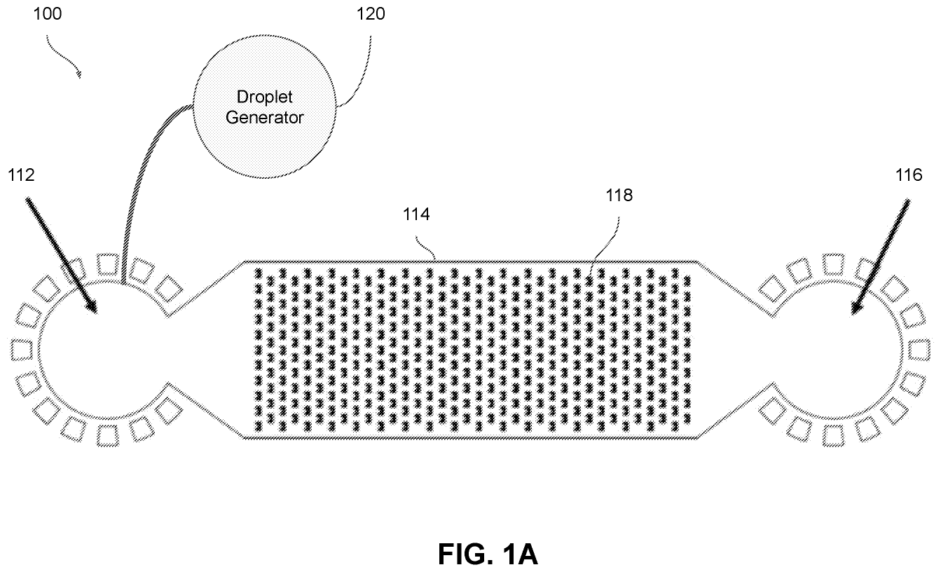

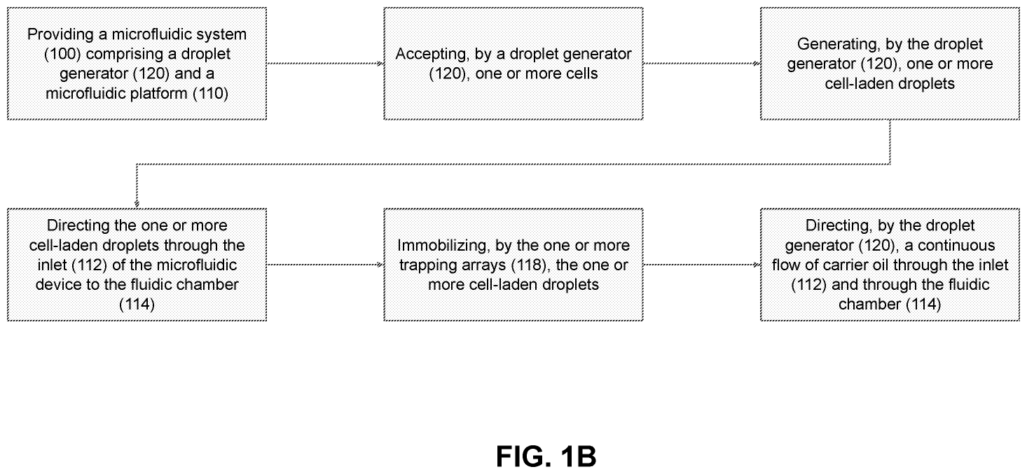

Referring now to FIG. 1A, the present invention features a microfluidic system (100) comprising a microfluidic platform (110). In some embodiments, the microfluidic platform (110) may comprise an inlet (112), a fluidic chamber (114) fluidly coupled to the inlet (112), comprising one or more trapping arrays (118), each trapping array comprising a plurality of pillars separated by gaps, and an outlet (116) fluidly coupled to the fluidic chamber (114). The microfluidic system (100) may further comprise a droplet generator (120) fluidly coupled to the inlet (112). The droplet generator (120) may accept one or more particles and output one or more particle-laden droplets, each particle-laden droplet comprising a particle of the one or more particles surrounded by an aqueous solution surrounded by a fluid. The one or more particle-laden droplets directed from the droplet generator (120), through the inlet (112), to the fluidic chamber (114) may be immobilized by the one or more trapping arrays (118). The droplet generator (120) may generate a continuous flow of fluid through the inlet (112) and through the fluidic chamber (114). The continuous flow of fluid may induce one or more microvortices within the one or more particle-laden droplets at the one or more trapping arrays (118). In some embodiments, the plurality of pillars may extend from an upper interior surface of the fluidic chamber (114), a lower interior surface of the fluidic chamber (114), or a combination thereof.

In some embodiments, one or more particles may comprise cells, beads, or any other material sized to fit within a droplet. In some embodiments, the continuous flow of fluid may comprise a carrier oil. In some embodiments, the aqueous solution comprises a cell culturing media, an isotonic media, or any media configured for cell growth and viability of the one or more cells.

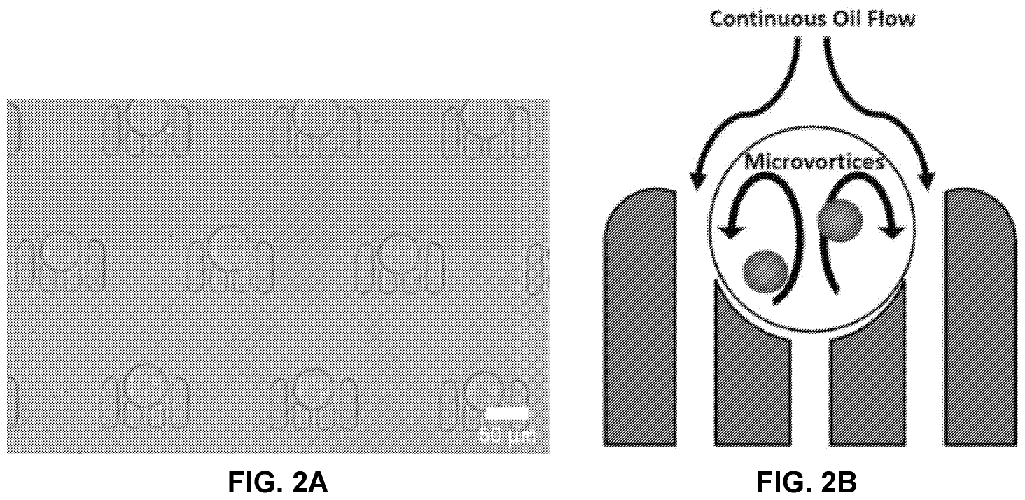

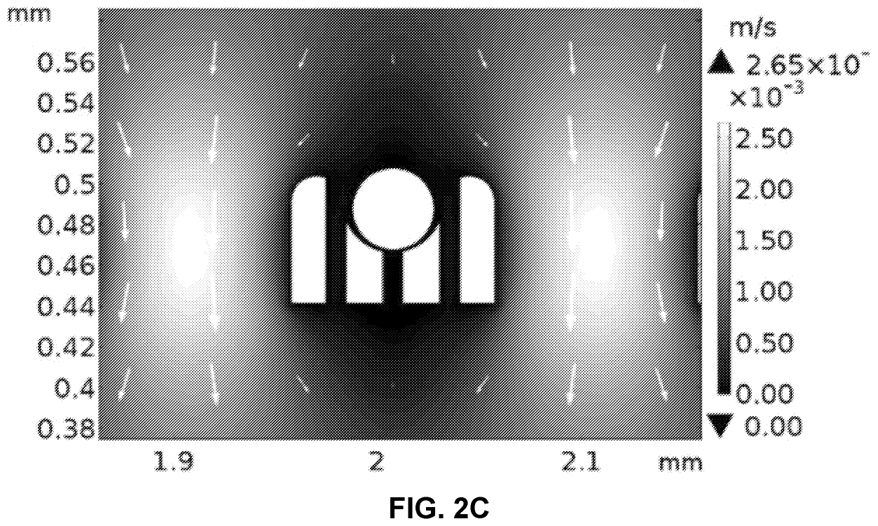

Referring now to FIG. 2, the present invention features a method comprising providing a microfluidic system (100) comprising a droplet generator (120) and a microfluidic platform (110) comprising an inlet (112), a fluidic chamber (114) fluidly coupled to the inlet (112), and an outlet (116) fluidly coupled to the fluidic chamber (114). The fluidic chamber (114) may comprise one or more trapping arrays (118), each trapping array comprising a plurality of pillars separated by gaps. The method may further comprise accepting, by a droplet generator (120), one or more cells, generating, by the droplet generator (120), one or more cell-laden droplets, each cell-laden droplet comprising a cell of the one or more cells surrounded by an aqueous solution, surrounded by a carrier oil, and directing the one or more cell-laden droplets through the inlet (112) of the microfluidic device to the fluidic chamber (114). The method may further comprise immobilizing, by the one or more trapping arrays (118), the one or more cell-laden droplets, and directing, by the droplet generator (120), a continuous flow of carrier oil through the inlet (112) and through the fluidic chamber (114). The continuous flow of carrier oil may induce one or more microvortices within the one or more cell-laden droplets at the one or more trapping arrays (118).

In some embodiments, the method may further comprise capturing, by an imaging apparatus, one or more images of the one or more cells in the one or more trapping arrays (118). In some embodiments, the imaging apparatus may comprise an epifluorescence microscope device. The imaging apparatus may capture the one or more images of the one or more cells by an Optical Projection Tomography (OPT) method. In some embodiments, the imaging apparatus may be coupled to a computing device comprising a processor configured to execute computer-readable instructions, and a memory component comprising computer-readable instructions for accepting the one or more images from the imaging apparatus, and generating a 3D reconstruction of each cell of the one or more cells based on the one or more images. In some embodiments, the movement of the one or more cells in the one or more trapping arrays (118) may allow for the OPT acquisition. The present invention may implement any imaging apparatus for continuous monitoring of the cells. It is this continuous monitoring which allows gathering cell footage, for instance, while they rotate, to generate the 3D images.

In some embodiments, the plurality of pillars may comprise polydimethylsiloxane (PDMS). In some embodiments, the plurality of pillars are separated by at least three gaps comprising a central gap and at least one lateral gap on each side of the central gap. For example, the trapping array may comprise 4 pillars and 3 gaps. In another embodiment, the trapping array may comprise 6 pillars and 5 gaps, where the central gap has two lateral gaps on each side. Without wishing to limit the present invention, the central gap can center the droplet within the trapping array. Without wishing to limit the present invention, each cell-laden droplet immobilized by the trapping array (118) can block the central gap such that the continuous flow of carrier oil flows through the lateral gaps around the cell-laden droplet.

In some embodiments, the plurality of pillars may be separated by about 1 to 25 μm gaps. In some embodiments, the plurality of pillars may be separated by about 5 to 15 μm gaps. In some embodiments, the plurality of pillars may be separated by about 10 μm gaps. In some embodiments, the plurality of pillars may be separated by about 2 to 3 μm gaps. In some embodiments, the plurality of pillars may be separated by about 2 to 50 μm gaps. In other words, the width of the gap may be about 1 to 50 μm gaps.

In some embodiments, each cell-laden droplet may have a diameter of about 20 to 70 μm. In some embodiments, each cell-laden droplet may have a diameter of about 40 to 60 μm. In some embodiments, each cell-laden droplet may have a diameter of about 50 μm. In some embodiments, each cell-laden droplet may have a diameter of about 10 to 5000 μm. In some embodiments, the plurality of pillars may be separated by gaps scaled to capture droplets of any diameter.

In some embodiments, if a trapping array of the one or more trapping arrays (118) is immobilizing a cell-laden droplet of the one or more cell-laden droplets, a subsequent cell-laden droplet may flow past the trapping array to a subsequent trapping array. In some embodiments, the one or more cells may comprise plant cells, mammalian cells, particles, or a combination thereof. In some embodiments, the aqueous solution may comprise a cell-culturing media. In some embodiments, the carrier oil may comprise HFE 7500 Engineering oil with 008-FluoroSurfactant or any other oil that could generate monodispersed droplets and stabilize droplets from coalescence.

In some embodiments, the plurality of pillars of the one or more trapping arrays (118) may comprise a plurality of inner pillars and a plurality of outer pillars. A length of the plurality of inner pillars may be less than a length of the plurality of outer pillars such that the plurality of pillars creates a pocket for immobilizing the cell-laden droplet. This is depicted in FIG. 2B. In some embodiments, the one or more gaps may act as microchannels for the continuous flow of carrier oil.

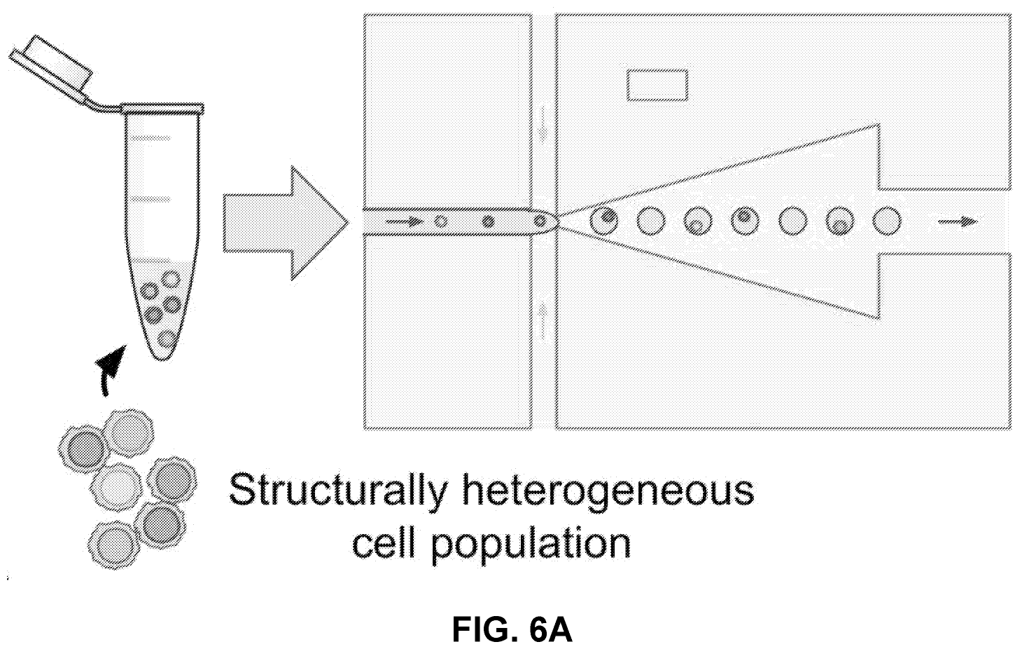

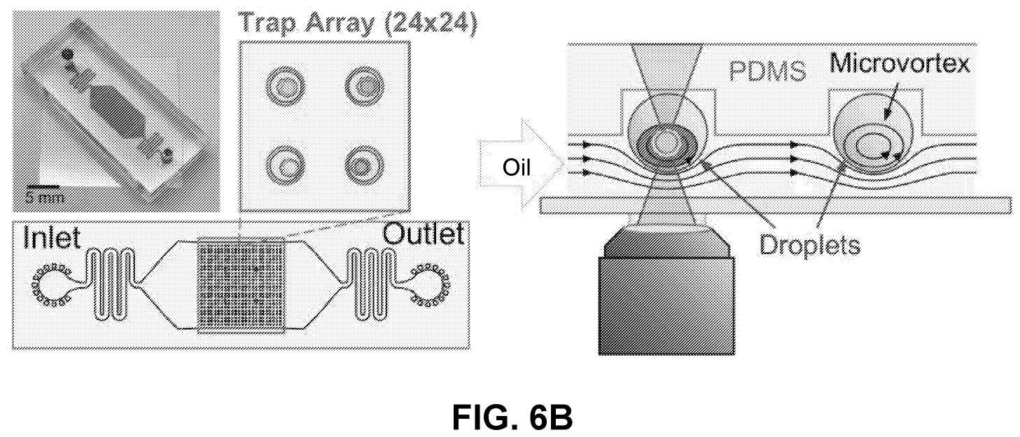

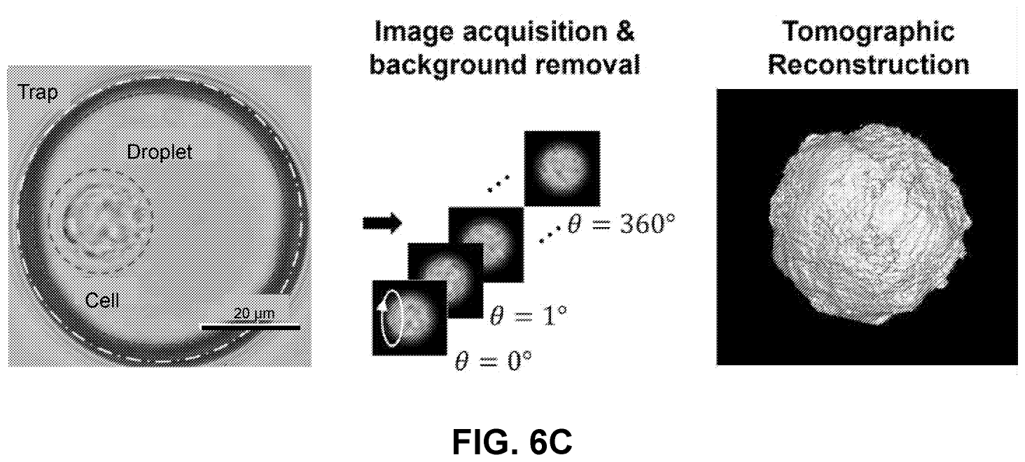

In alternative embodiments, the trapping arrays (118) may comprise one or more pockets disposed in an upper interior surface of the fluidic chamber (114), a lower or bottom interior surface of the fluidic chamber (114), or a combination thereof. As shown in FIG. 6B, pockets disposed in the upper interior surface may be referred to as inverted microwells. Pockets disposed in the bottom interior surface may be referred to as microwells.The trapping array (118) may comprise a plurality of pockets. In some embodiments, each pocket is sized to capture and retain one droplet. The continuous flow of fluid in the channel can induce microvortices within the droplet that is in the pocket. Without wishing to limit the present invention, the continuous flow of carrier oil or fluid has a flow rate such that one or more microvortices in the particle-laden droplet induce spinning of the particle within the particle-laden droplet or orbiting of the particle around a point within the particle-laden droplet.

The present invention features a microfluidic platform that is not only capable of immobilizing cell-laden droplets but also leveraging the shear stress at the liquid-liquid interface to induce microvortices of well-defined shape and periodicity for cell pairing applications.

The trapping arrays were fabricated by soft lithography, where the specially designed trap consists of PDMS pillars separated by 10 m gaps as illustrated in FIG. 1A. A separate microdevice was used to generate the 50 m water-in-oil droplets before loading into the trapping arrays. After the aqueous droplets surrounded by immiscible carrier oil were immobilized by the trapping arrays, the carrier oil flow would be continuously delivered into the device. Owing to the uniquely designed traps with gaps, the continuous oil would bilaterally flow around the immobilized droplets that exerts shear stress at the liquid-liquid interface to induce the microvortices (FIGS. 2A-2B). Once a trap is occupied with a droplet, any free droplets would be preferentially diverted and flow into the subsequent traps due to lower resistance, as confirmed by the numerical simulation (FIG. 2C). Thus, the trapping arrays of the present invention were able to consistently achieve 100% occupancy for immobilizing droplets. In some embodiments, the ratio of the size of the droplet to the size of the particle therein may determine whether the microvortices induce spinning or orbiting of the particle in the droplet. For example, a smaller ratio may result in spinning of the particle within the droplet, while a larger ratio may result in orbiting of the particle within the droplet about its center.

With regards to the proposed droplet trap microvortices system, one can estimate the intra-droplet vortex periodicity by assuming a droplet of viscosity ηᵢ and radius R₀, immersed in an unbounded fluid of viscosity ηₒ. Setting −U₀\hat{z} far from the liquid-liquid interface, the solution of the steady-state Stokes equations leads to the Hadamard-Rybczynski velocity fields for the inner and outer phases. Closed particle pathlines are periodic on both r(t) and θ(t) polar coordinates, with period T, which is given by:

$$T = \frac{2 τ}{\sqrt{{( {r}_{0} / {R}_{0} )}^{2} - 1}} [ K ( \frac{{r}_{0}^{2}}{{R}_{0}^{2} - {r}_{0}^{2}} ) - F ( ϕ , \frac{{r}_{0}^{2}}{{R}_{0}^{2} - {r}_{0}^{2}} ) ] = τ f ( {r}_{0} , {R}_{0} ) , \qquad ( 1 )$$

$$τ = \frac{2 R 0}{{U}_{0}} ( 1 + \frac{{η}_{i}}{{η}_{0}} )$$

where r₀ is the vortex starting point, K(m) and F(ϕ, m) are the complete and incomplete elliptic integrals of the first kind, respectively, with ϕ=sin⁻[(R₀²−r₀²)/r₀²], and τ is a characteristic time constant.

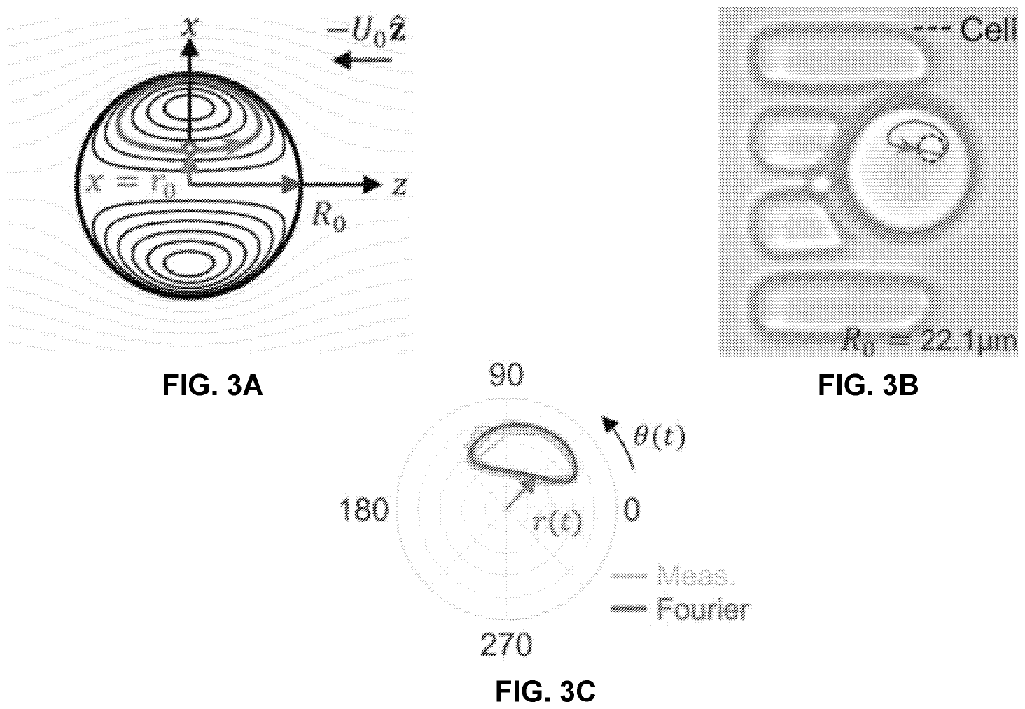

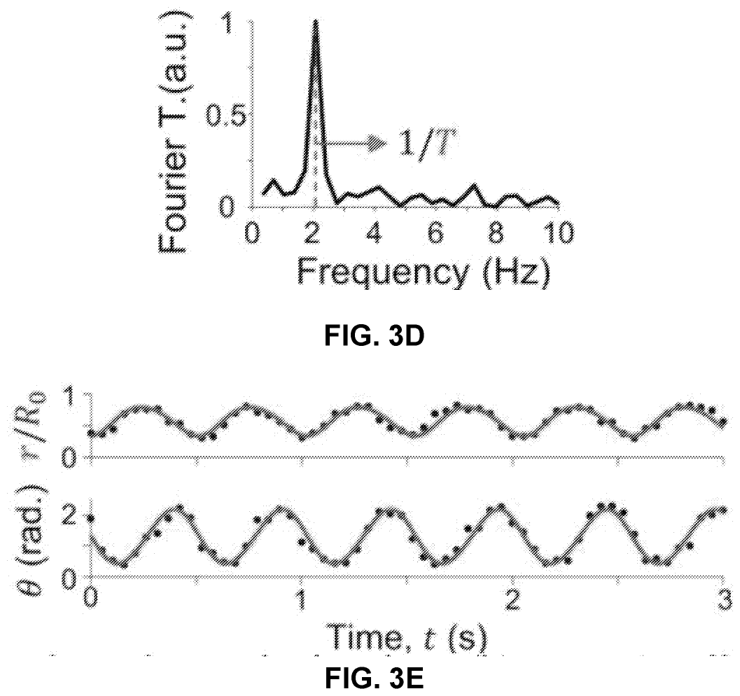

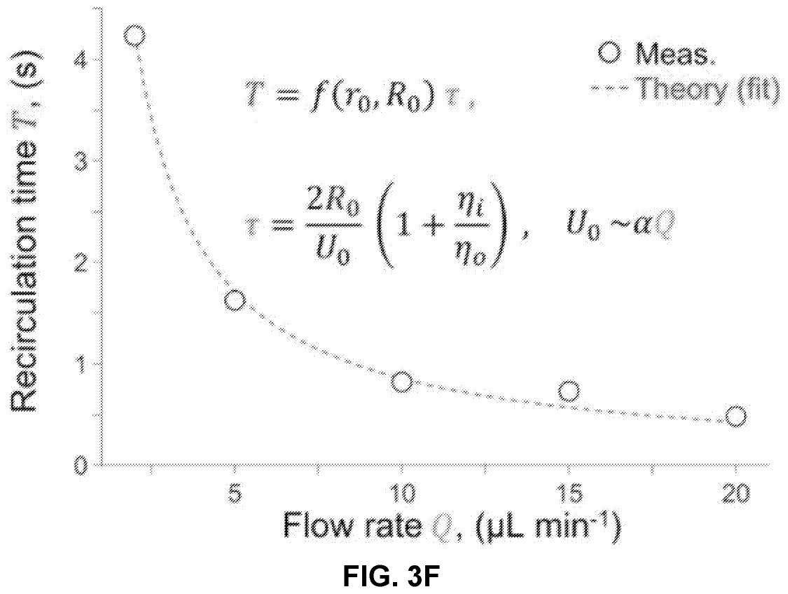

A bright-field image in FIG. 3B shows a circulating K562 cell upon externally applying 20 μLmin⁻¹. Particle Tracking Velocimetry (PTV) reveals the hemisphere-like path traced by the cell in FIG. 3C, which has been represented by the first two Fourier harmonics (line). A Fourier transform of r(t) in FIG. 3D illustrates the periodicity of the coordinate time-course data (FIG. 3E), peaking at T for the first harmonic. By comparing the measured T v.s flow rate Q, an inverse relationship arises, as predicted from the characteristic time τ in eq. (1). Because the external flow speed U₀ appears in eq. (1), rather than Q, a functional fit was performed, justified by the fact that for Stokes flow, U₀˜αQ. Values of α=1.5×10⁶ m⁻² with R²=0.996 support the hypothesis that Hadamard-Rybczynski—like vortices arise in droplets; further, α-values are plausible, as 20 μL min⁻¹ translates into U₀˜500 μms⁻¹.

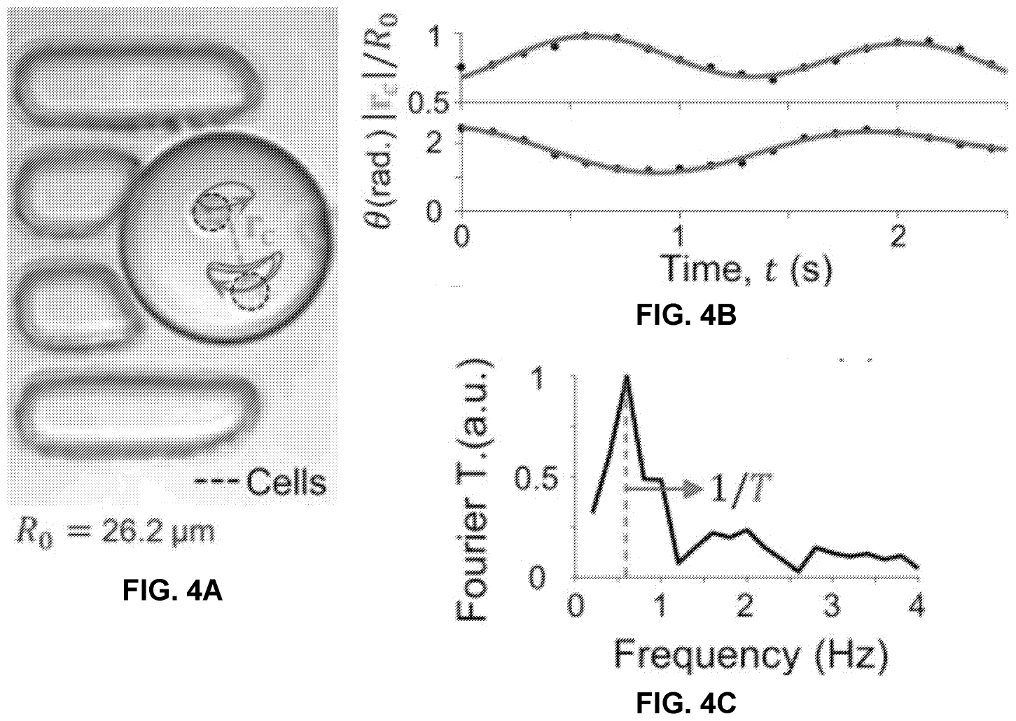

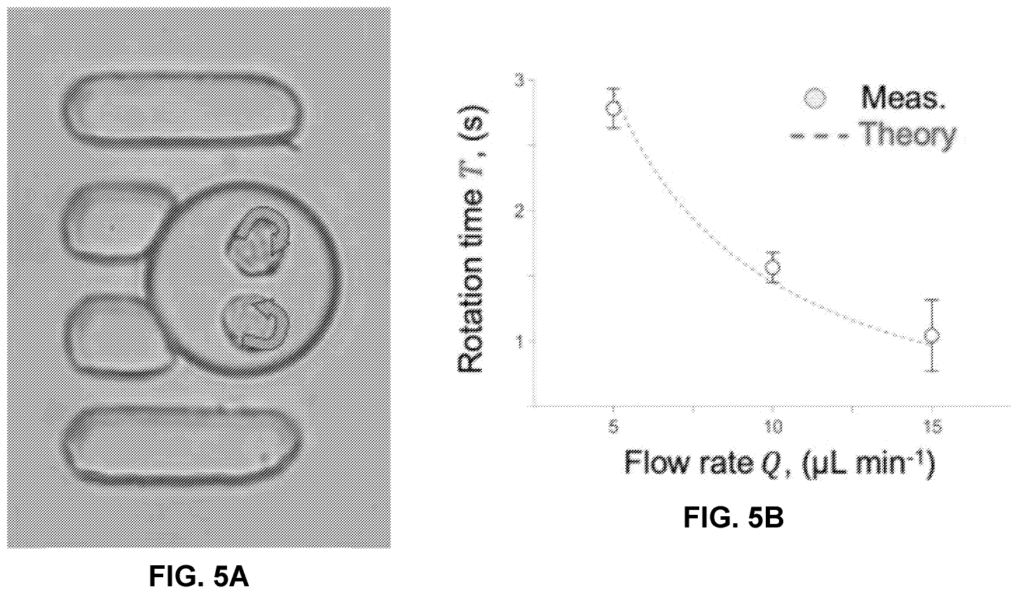

In FIG. 4A-4C shows that the microvortex analysis in FIGS. 3A-3E is applicable to two-particle systems. Monitoring of the compound vector r_{c}=r₁−r₂ components reveals the time-course of cell-to-cell distance (FIG. 4B), which retains periodicity albeit with asymmetric vortex patterns (FIG. 3C). In addition to the aforementioned cell orbiting driven by the microvortices, the self-rotational motion of cells is discovered at the center of microvortices when the droplets are of optimal size and external flow rate. The self-rotational time also exhibits an inverse relationship with respect to the external flow rate (FIG. 5A-5B), which is in accordance with the analytical analysis.

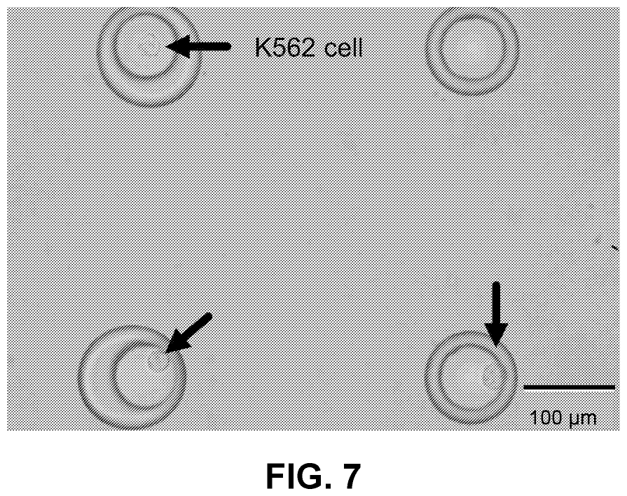

In FIG. 6A-6B and FIG. 7A-7B, the present invention features a method providing a microfluidic system comprising two inlets, a fluidic chamber fluidly coupled to the inlets, and two outlets fluidly coupled to the fluidic chamber. The fluidic chamber may comprise one or more trapping arrays, each trapping array comprising a plurality of inverted microwells. The method may further comprise accepting, by a droplet generator, one or more cells, generating, by the droplet generator, one or more cell-laden droplets, each cell-laden droplet comprising a cell of the one or more cells surrounded by an aqueous solution, surrounded by a carrier oil, and directing the one or more cell-laden droplets through two inlets of the microfluidic device to the fluidic chamber. The method may further comprise immobilizing, by the one or more trapping arrays, the one or more cell-laden droplets, and directing, by the droplet generator, a continuous flow of carrier oil through two inlets and through the fluidic chamber. The continuous flow of carrier oil may induce one or more microvortices within the one or more cell-laden droplets at the one or more trapping arrays.

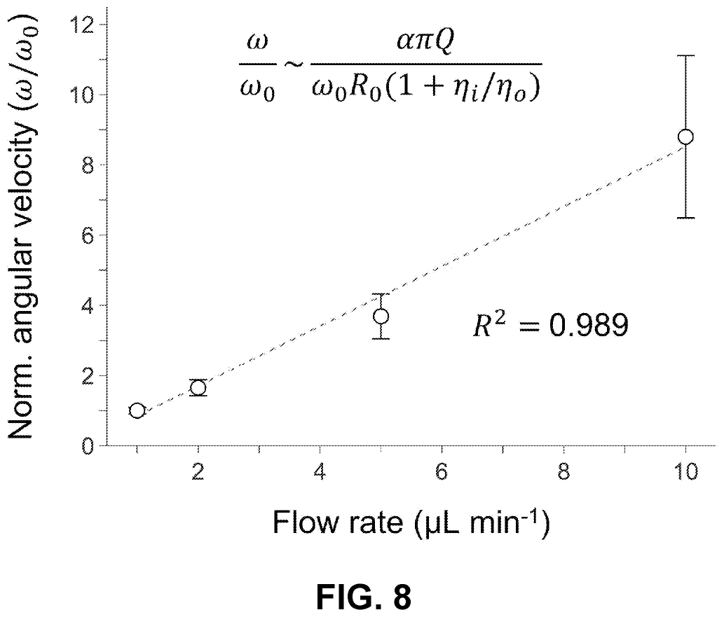

Therefore, the main results—vortex recirculation time control, and periodic modulation of cell-to-cell distance and self-rotation through passive hydrodynamics inside compartmentalized microfluidic units—are applicable to droplet-based immuno-analysis, where the transient scanning interaction and proximity between effector and target cell need to be deterministically regulated while retaining pairwise-correlated information to facilitate the study of immunological synapses. Another main result is the fact that the microvortices can be used to self-rotate (spin) cells on their axis, which ultimately can be used to study cell morphology, as shown in FIG. 8.

One intended application for this technology would be the immune analysis between effector and target cell, including chimeric antigen receptor (CAR)-T and CD19-presenting cell interactions using cytokine capture beads to assess the activation through interferon-gamma secretion. In addition, the periodic rotation of cells within a stationary droplet enables the ability to investigate the cell morphology including 3-D surface topography, receptor distribution, and surface markers at scale.

The present invention features a method for 3D fluorescent imaging of live, non-adherent single cells encapsulated in picoliter droplets using Optical Projection Tomography (OPT) enabled by droplet microvortices. OPT relies on the capability to manipulate the 3D orientation of an image object in relation to the optical imaging axis to produce 2D X-ray-like pseudo-projections. In single-cell OPT, rotation methods include microcapillary rotation and electrorotation, which are effective but have low throughput. To use microcapillaries, cells need to be embedded in the cytotoxic thixotropic index-matched gel, while electrorotation requires solutions that are different from normal isotonic media, thus limiting the biological assays that can be conducted during observation. In contrast, the present invention features a microfluidic droplet trap array that leverages the flow-induced interfacial shear to generate intra-droplet microvortices (FIGS. 6A-6C and 7). These microvortices then mobilize the encapsulated cells at an angular velocity determined by the flow rate (FIG. 8). This strategy allows observation of cells encapsulated inside droplets of conventional, non-toxic isotonic buffer, and facilitates scalable OPT acquisition by the simultaneous spinning of hundreds of cells.

FIG. 8 shows the dependence of cell angular velocity (ω) with the applied bulk flow rate, normalized by the baseline angular velocity (ω₀) when Q=1 μLmin⁻¹. To explain the linear relationship in FIG. 8 (R²=0.989), the recirculation timescale (τ) of an infinitesimally small particle following the closed-loop vortex streamlines of a Hadamard-Rybczynski field was derived. It was found that T is given by τ˜2R₀/U₀(1+ηᵢ/ηₒ), where R₀ is the droplet radius, U₀ the bulk flow velocity and ηᵢ/ηₒ the ratio of inner and outer phase viscosities. Assuming the cell rotation period is T˜τ, and U₀ is proportional to the applied flow rate (U₀˜αQ), ω becomes ω(Q)˜απQ/R₀(1+ηᵢ/ηₒ).

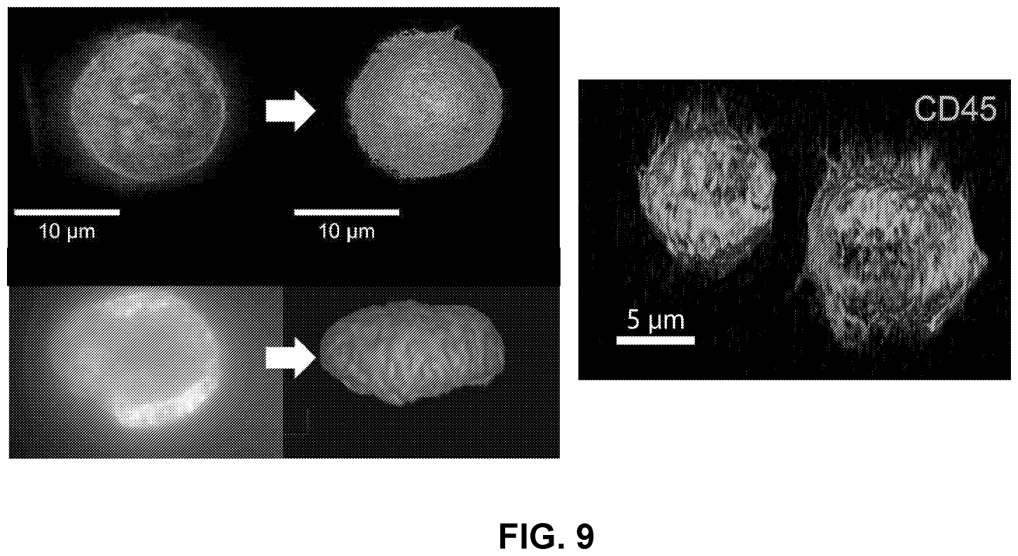

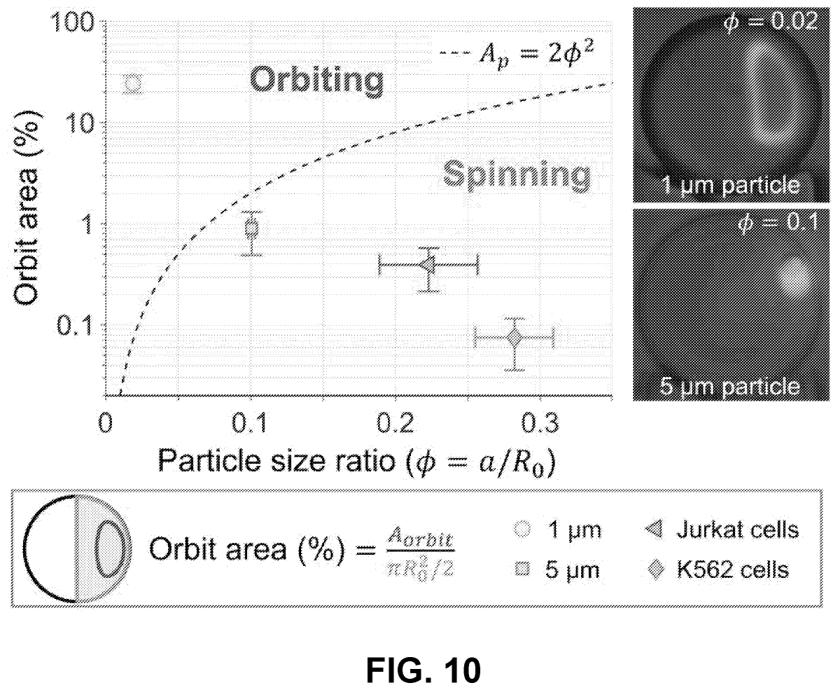

FIG. 9 demonstrates a 3D fluorescence reconstruction of a fully suspended K562 cell (CD45+, Alexa Fluor 488). Stained cells were suspended in 1× DPBS solution with 16% Optiprep, followed by droplet encapsulation (R₀=30 μm, channel height: 40 μm) using a previously described microdevice. After droplet loading into microfluidic traps, a flow rate of 1 μLmin⁻¹ was used to produce rotation rates of 4.2 rpm (N=550−600, 25 ms exposure images acquired using a 100×, NA=1.3 objective). The OPT routine is based on a reported direct inversion algorithm for focal plane scanned images, which helped reduce noise from out-of-focus light. The method further allowed the reconstruction of the K562 nuclei (NucBlue™, FIG. 9), demonstrating that intracellular structures can be recovered with isotropic resolution (xy resolution of 79 nm/px).

Droplet microvortices provide a unique strategy for scalable, single-cell rotation with no special media requirements. Flow-controlled, linear cell rotation allowed 360°-imaging of non-adherent cells with high lateral resolution using simple epifluorescence microscopy. This is in contrast with complex optical tomographic setups, and optical sectioning techniques with poor axial resolution due to the missing cone problem. The present invention is configured to identify surface and intracellular characteristics of immune cells in their natural suspended phenotype. Additionally, it is capable of detecting morphological markers under conditions affecting their immune effector functions, including immunological activation and suppression.

The present invention features a water-in-oil droplet microfluidic trap array capable of modulating the distance between co-encapsulated cell pairs through microvortex formation. Vortex shape and periodicity are directly controlled by the continuous phase flow rate. Explicit equations for the recirculation time inside droplet microvortices were derived by approximating the velocity fields through analytic solutions for the flow inside and outside of a spherical droplet. Comparison of these expressions against Particle Tracking Velocimetry (PTV) measurements of K562 (leukemia) cells circulating inside 50 μm droplets showed excellent theoretical agreement.

### EXAMPLE

The following is a non-limiting example of the present invention. It is to be understood that said example is not intended to limit the present invention in any way. Equivalents or substitutes are within the scope of the present invention.

Trap arrays with 40 μm height were fabricated by soft-lithography. PDMS pillars were separated by 10 μm to allow bilateral oil flow around trapped droplets. A separate microdevice was used to generate the 50 μm water-in-oil droplets. The dispersed phase consisted of 1× PBS, 16% Optiprep™ and 0.01% Triton™ X-100, and HFE7500 oil with fluorosurfactant was selected as the continuous phase. Microvortices inside droplets were induced by controlling the continuous phase flow rate between 2-20 μLmin−1, and PTV was used to measure the particle motion.

The droplet of viscosity ηᵢ was approximated with a circle of radius R₀, immersed in an unbounded fluid of viscosity ηₒ FIG. 3A). Setting −U₀\hat{z} far from the liquid-liquid interface, the solution of the steady-state Stokes equations led to the Hadamard-Rybczynski velocity fields for the inner and outer phases. Closed particle pathlines were periodic on both r(t) and θ(t) coordinates, with period T, which is given by equation (1), listed above.

A bright-field image in FIG. 3B shows a circulating K562 cell upon externally applying 20 μLmin⁻¹. Particle Tracking Velocimetry (PTV) revealed the hemisphere-like path traced by the cell in FIG. 3C, which has been represented by the first two Fourier harmonics (line). A Fourier transform of r(t) in FIG. 3D illustrates the periodicity of the coordinate time-course data (FIG. 3E), peaking at T for the first harmonic. By comparing the measured T v.s flow rate Q, an inverse relationship arose, as predicted from the characteristic time τ in eq. (1). Because the external flow speed U₀ appeared in the prior equation, rather than Q, a functional fit was performed, justified by the fact that for Stokes flow, U₀˜αQ. Values of α=1.5×10⁶ m⁻² with R²=0.996 support the hypothesis that Hadamard-Rybczynski-like vortices arise in droplets; further, α-values are plausible, as 20 μL min⁻¹ translates into U₀˜500 μm s⁻¹.

The microvortex analysis in FIGS. 3A-3F was applicable to two-particle systems. Monitoring of the compound vector r_{c}=r₁−r₂ components revealed the time-course of cell-to-cell distance, which retained periodicity albeit with asymmetric vortex patterns. Therefore, the main results—vortex recirculation time control, and periodic modulation of cell-to-cell distance through passive hydrodynamics inside compartmentalized microfluidic units—were applicable to droplet-based immuno-analysis, where the transient scanning interaction and proximity between effector and target cell needed to be deterministically regulated while retaining pairwise-correlated information to facilitate the study of immunological synapses.

Through experimental and theoretical understanding of flow patterns in droplets, the feasibility of modulating the position of encapsulated cells was demonstrated, thus providing a critical tool for studying dynamic cell-cell communication. This general particle manipulation approach is thereby applicable to a broad range of applications including drug screening, immunology, immunotherapy, and tissue engineering.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A method comprising:
a. providing a microfluidic system (100) comprising a droplet generator (120) and a microfluidic platform (110) comprising an inlet (112), a fluidic chamber (114) fluidly coupled to the inlet (112), and an outlet (116) fluidly coupled to the fluidic chamber (114), wherein the fluidic chamber (114) comprises one or more trapping arrays (118);
b. accepting, by a droplet generator (120), one or more particles;
c. generating, by the droplet generator (120), one or more particle-laden droplets, each particle-laden droplet comprising a particle of the one or more particles surrounded by an aqueous solution, surrounded by a carrier oil;
d. directing the one or more particle-laden droplets through the inlet (112) of the microfluidic device to the fluidic chamber (114);
e. immobilizing, by the one or more trapping arrays (118), the one or more particle-laden droplets; and
f. directing, by the droplet generator (120), a continuous flow of carrier oil through the inlet (112) and through the fluidic chamber (114);
wherein the continuous flow of carrier oil induces one or more microvortices within the one or more particle-laden droplets at the one or more trapping arrays (118).

2. The method of claim 1, wherein the one or more trapping arrays (118) comprise a plurality of pillars separated by gaps.

3. The method of claim 2, wherein the plurality of pillars are separated by 2 to 50 μm gaps.

4. The method of claim 2, wherein the plurality of pillars are separated by at least three gaps comprising a central gap and at least one lateral gap on each side of the central gap, wherein each particle-laden droplet immobilized by the trapping array (118) blocks the central gap such that the continuous flow of carrier oil flows through the lateral gaps around the particle-laden droplet.

5. The method of claim 1, wherein the one or more trapping arrays (118) comprise one or more pockets disposed in an upper interior surface of the fluidic chamber (114), a bottom interior surface of the fluidic chamber (114), or a combination thereof.

6. The method of claim 1, wherein the continuous flow of carrier oil has a flow rate such that the one or more microvortices induce, for each particle-laden droplet of the one or more particle-laden droplets, spinning of the particle within the particle-laden droplet or orbiting of the particle around a point within the particle-laden droplet.

7. A microfluidic system (100) comprising:
a. a microfluidic platform (110) comprising:
i. an inlet (112);
ii. a fluidic chamber (114) fluidly coupled to the inlet (112), comprising one or more trapping arrays (118), each trapping array comprising a plurality of pillars separated by gaps; and
iii. an outlet (116) fluidly coupled to the fluidic chamber (114); and
b. a droplet generator (120) fluidly coupled to the inlet (112);
wherein the droplet generator (120) accepts one or more cells and outputs one or more cell-laden droplets, each cell-laden droplet comprising a cell of the one or more cells surrounded by an aqueous solution surrounded by a carrier oil;
wherein the one or more cell-laden droplets directed from the droplet generator (120), through the inlet (112), to the fluidic chamber (114) are immobilized by the one or more trapping arrays (118);
wherein the droplet generator (120) generates a continuous flow of carrier oil through the inlet (112) and through the fluidic chamber (114);
wherein the continuous flow of carrier oil induces one or more microvortices within the one or more cell-laden droplets at the one or more trapping arrays (118).

8. The system (100) of claim 7, wherein the plurality of pillars extend from an upper interior surface of the fluidic chamber (114), a lower interior surface of the fluidic chamber (114), or a combination thereof.

9. The system (100) of claim 7, wherein the plurality of pillars are separated by 2 to 50 μm gaps.

10. The system (100) of claim 7, wherein each cell-laden droplet has a diameter of 10 to 5000 μm.

11. The system (100) of claim 7, wherein if a trapping array of the one or more trapping arrays (118) is immobilizing a cell-laden droplet of the one or more cell-laden droplets, a subsequent cell-laden droplet flows past the trapping array to a subsequent trapping array.

12. The system (100) of claim 7, wherein the one or more cells comprise plant cells, mammalian cells, particles, or a combination thereof.

13. The system (100) of claim 7, wherein the aqueous solution comprises a cell culturing media, an isotonic media, or any media configured for cell growth and viability of the one or more cells.

14. The system (100) of claim 7 further comprising an imaging apparatus optically coupled to the fluidic chamber (114), configured to capture one or more images of the one or more cells in the one or more trapping arrays (118).

15. The system (100) of claim 7, wherein, for each trapping array of the one or more trapping arrays (118), the plurality of pillars are separated by at least three gaps comprising a central gap and at least one lateral gap on each side of the central gap, wherein each cell-laden droplet immobilized by the trapping array (118) blocks the central gap such that the continuous flow of carrier oil flows through the lateral gaps around the cell-laden droplet.

16. The system (100) of claim 7, wherein the continuous flow of carrier oil has a flow rate such that the one or more microvortices induce, for each cell-laden droplet of the one or more cell-laden droplets, spinning of the cell within the cell-laden droplet or orbiting of the cell around a point within the cell-laden droplet.

17. A method comprising:
a. providing a microfluidic system (100) comprising a droplet generator (120) and a microfluidic platform (110) comprising an inlet (112), a fluidic chamber (114) fluidly coupled to the inlet (112), and an outlet (116) fluidly coupled to the fluidic chamber (114);
wherein the fluidic chamber (114) comprises one or more trapping arrays (118), each trapping array comprising a plurality of pillars separated by gaps;
wherein, for each trapping array of the one or more trapping arrays (118), the plurality of pillars are separated by at least three gaps comprising a central gap and at least one lateral gap on each side of the central gap, wherein each cell-laden droplet immobilized by the trapping array (118) blocks the central gap such that the continuous flow of carrier oil flows through the lateral gaps around the cell-laden droplet;
b. accepting, by a droplet generator (120), one or more cells;
c. generating, by the droplet generator (120), one or more cell-laden droplets, each cell-laden droplet comprising a cell of the one or more cells surrounded by an aqueous solution, surrounded by a carrier oil;
d. directing the one or more cell-laden droplets through the inlet (112) of the microfluidic device to the fluidic chamber (114);
e. immobilizing, by the one or more trapping arrays (118), the one or more cell-laden droplets; and
f. directing, by the droplet generator (120), a continuous flow of carrier oil through the inlet (112) and through the fluidic chamber (114);
wherein the continuous flow of carrier oil induces one or more microvortices within the one or more cell-laden droplets at the one or more trapping arrays (118);
wherein the continuous flow of carrier oil has a flow rate such that the one or more microvortices induce, for each cell-laden droplet of the one or more cell-laden droplets, spinning of the cell within the cell-laden droplet or orbiting of the cell around a point within the cell-laden droplet.

18. The method of claim 17, wherein the plurality of pillars extend from an upper interior surface of the fluidic chamber (114), a lower interior surface of the fluidic chamber (114), or a combination thereof.

19. The method of claim 17, wherein if a trapping array of the one or more trapping arrays (118) is immobilizing a cell-laden droplet of the one or more cell-laden droplets, a subsequent cell-laden droplet flows past the trapping array to a subsequent trapping array.

20. The method of claim 17 further comprising:
a. capturing, by an imaging apparatus, one or more images of the one or more cells in the one or more trapping arrays (118); and
b. generating, by a computing device communicatively coupled to the imaging apparatus, a 3D reconstruction of each cell of the one or more cells based on the one or more images.
